(19) United States
(12) Patent Application Publication
(10) Pub. No.: US 2022/0125700 A1
(43) Pub. Date: Apr. 28, 2022

(54) BIO-DEGRADABLE MICROCAPSULES

(71) Applicant: INTERNATIONAL FLAVORS & FRAGRANCES INC., Union Beach, NJ (US)

(72) Inventors: Yabin LEI, Union Beach, NJ (US); Li XU, Union Beach, NJ (US)

(21) Appl. No.: 17/570,498

(22) Filed: Jan. 7, 2022

Related U.S. Application Data
(63) Continuation-in-part of application No. 16/458,273, filed on Jul. 1, 2019, now Pat. No. 11,224,569.
Application No. 16/458,273 is a continuation of application No. 15/722,022, filed on Oct. 2, 2017, now Pat. No. 10,369,094.
Application No. 15/722,022 is a continuation-in-part of application No. 14/414,376, filed on Jan. 12, 2015, now Pat. No. 9,777,244.

(51) Int. Cl. A61K 8/81; A61K 9/50; A61K 8/06; A61Q 5/02; A61Q 5/06; A61Q 5/12; A61Q 15/00; A61Q 17/00; A61Q 19/10; A61K 8/11; C11D 3/50

(52) U.S. Cl. CPC A61K 8/8152; A61K 2800/624; A61K 9/5026; A61K 8/068; A61Q 5/02; A61Q 5/06; A61Q 5/12; A61Q 15/00; A61Q 17/005; A61Q 19/10; A61K 8/11; C11D 3/50; C11D 3/505; A61K 2800/5424; A61K 2800/56; A61K 2800/412; A61K 2800/5426; A61K 8/8147

(57) ABSTRACT

Bio-degradable microcapsules with a fragrance or odorant encapsulated therein during the polymerization process are provided. The bio-degradable microcapsules are of use in fabric care or personal care formulations.

### INTRODUCTION

This application is a Continuation-in-Part application of U.S. patent application Ser. No. 16/458,273, filed Jul. 1, 2019, which is a Continuation application of U.S. patent application Ser. No. 15/722,022, filed Oct. 2, 2017, now U.S. Pat. No. 10,369,094, which is a Continuation-in-Part application of U.S. patent application Ser. No. 14/414,376, filed Jan. 12, 2015, now U.S. Pat. No. 9,777,244, the contents of which are incorporated herein by reference in their entireties.

### BACKGROUND OF THE INVENTION

For protection of valuable chemical compounds such as fragrances or odorants, the use of encapsulation is increasingly being considered or is already being practiced. Encapsulation refers to processes whereby an active ingredient is placed into a stabilized form to allow it to be conveniently stored, or protected from unfavorable conditions, until needed. The release of active ingredient from the protected form may be rapid (such as by crushing, or by ingestion), or gradual (such as by dissolution, diffusion, or bio-degradation). In this manner it is possible to maximize the effectiveness of the active ingredient by ensuring that it is released at the proper time.

The term “microcapsule” has been used to describe small particles or beads, which range in size from less than one micron, up to several millimeters, which may contain a wide variety of active ingredients (Thies (1994) Today's Chemist November p. 40; Goodwin (1974) Chemtech Magazine October p623-626). Microcapsules can be divided into two broad groups: (1) “Aggregate” type microcapsules having the active ingredient dispersed uniformly throughout a continuous matrix. The matrix may be a solid dry polymer or a gel swollen with solvent. In the case where the gel is swollen with water, the term “hydrogel” is applied. Hydrogel encapsulation systems of this type have been described and are generally based on cross-linked forms of water-soluble polymers such as alginate, gelatin, pectin, agar, gellan, or starch (Sanderson, et al. (1989) Cereal Foods World 34(12):993-998). (2) “Mononuclear” microcapsules, on the other hand, are composed of materials that show a true “shell-core” morphology. These are similar to an egg in that they have a solid shell or flexible membrane surrounding a core which may be a liquid, a solid, or even a gel.

“Bio-degradable” or “bio-degradability” refers to substance that is capable of undergoing and/or does undergo microbial and/or biological degradation. The bio-degradability of a substance is assessed by passing one or more of the following tests including: a respirometry bio-degradation method in aquatic media, available from Organization for Economic Cooperation and Development (OECD), International Organization for Standardization (ISO) and the American Society for testing and Material (ASTM) tests including, but not limited to OECD 301F or 310 (Ready bio-degradation), OECD 302 (inherent bio-degradation), ISO 17556 (solid stimulation studies), ISO 14851 (fresh water stimulation studies), ISO 18830 (marine sediment stimulation studies), OECD 307 (soil stimulation studies), OECD 308 (sediment stimulation studies), and OECD 309 (water stimulation studies). For example, substance may be deemed bio-degradable when at least 60% of the substance is degraded within 60 days according to OECD301F test.

### SUMMARY OF THE INVENTION

This invention includes bio-degradable core-shell microcapsules, a consumer product (e.g., a laundry care, personal care, therapeutic, cosmetic or cosmeceutical product) containing the bio-degradable core-shell microcapsules and a method for preparing the microcapsules. The bio-degradable core-shell microcapsules are composed of an active material, e.g., a fragrance or odorant, encapsulated in a shell composed of sodium alginate, chitosan (e.g., fungal-derived chitosan or a derivative thereof), tannic acid, or a combination thereof. In some aspects, the active material is a fragrance including at least one, at least two, at least three, at least four, at least five, at least six, at least seven, at least eight, at least nine or at least ten fragrance ingredients. In other aspects, the microcapsules shell is inherently bio-degradable, primarily bio-degradable or ultimately bio-degradable and/or has a bio-degradability of at least 20%, 30%, 40%, 50%, 60%, 65%, 70%, 75%, 80%, 85%, 90%, 95% or 98%, within 60 days according to OECD301F or OECD310. Ideally, the active material is a fragrance oil and has a total loading that ranges from 10% to 40%.

The method of producing the bio-degradable core-shell microcapsules involves the steps of (a) preparing an aqueous phase comprising sodium alginate, chitosan, tannic acid, or a combination thereof; (b) preparing an oil phase containing an active material; (c) emulsifying the aqueous phase of (a) with the oil phase of (b) to produce an emulsion; (d) polymerizing the emulsion to produce bio-degradable core-shell microcapsules with the active material encapsulated therein; and (e) curing the bio-degradable core-shell microcapsules at a temperature in the range of 55° C. to 95° C.

Laundry care, personal care, therapeutic, cosmetic or cosmeceutical products containing the bio-degradable core-shell microcapsules of this invention are also provided.

### DETAILED DESCRIPTION OF THE INVENTION

This invention relates to bio-degradable core-shell microcapsules as carriers for fragrances or odorants, the production thereof and the use of the carrier in providing a fragrance or odorant to a fabric care product or personal care product, e.g., hair conditioner/shampoo, body lotion, or hair refresher, as well as washing or cleaning compositions for laundry and surface treatment. The bio-degradable core-shell microcapsules of this invention are composed of a fragrance or odorant encapsulated in a shell, wherein a bio-degradable polymer is incorporated into the shell during preparation of the microcapsule.

A fragrance or odorant is understood to mean all organic substances which have a desired olfactory property and are essentially nontoxic. This includes all fragrances or odorants used customarily in washing or cleaning compositions or in perfumery and includes fragrance mixtures or blends. A fragrance or odorant may be a compound of natural, semisynthetic or synthetic origin. Preferred fragrances or odorants can be assigned to the substance classes of the hydrocarbons, aldehydes or esters. The fragrances or odorants also include natural extracts and/or essences which may include complex mixtures of constituents, such as orange oil, lemon oil, rose extract, lavender, musk, patchouli, balsam essence, sandalwood oil, pine oil and cedar oil.

Non-limiting examples of synthetic and semisynthetic fragrances or odorants are: 7-acetyl-1,2,3,4,5,6,7,8-octahydro-1,1,6,7-tetramethylnaphthalene, α-ionone, β-ionone, γ-ionone, α-isomethylionone, methyl cedryl ketone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl)propionaldehyde, ethylvanillin, heliotropin, hexylcinnamaldehyde, amylcinnamaldehyde, 2-methyl-2-(isopropylphenyl)propionaldehyde, coumarin, decalactone-γ, cyclopentadecanolide, 16-hydroxy-9-hexadecenolactone, 1,3,4,6,7,8-hexahydro-4,6,6,7,8,8-hexamethylcyclopenta-γ-2-benzopyran, β-naphthol methyl ether, ambroxan, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate and tert-butylcyclohexyl acetate.

Further examples of fragrances or odorants of use in this invention are described, for example, in U.S. Pat. Nos. 6,143,707; 6,703,011; and 5,089,162; EP 1 360 270 and WO 2009/027957.

Other fragrances are essential oils, resinoids and resins from a multitude of sources, such as peru balsam, olibanum resinoid, styrax, labdanum resin, nutmeg, cassia oil, benzoin resin, coriander and lavandin. Further suitable fragrances are: phenyl ethyl alcohol, terpineol, linalool, linalyl acetate, geraniol, nerol, 2-(1,1-dimethylethyl)cyclohexanol acetate, benzyl acetate and eugenol.

The fragrances provided in Table 1 are amongst those suitable for inclusion within the microcapsule of the present invention.

TABLE 1

| No. | Perfume Ingredient | ClogP |
|---|---|---|
| 1 | Allyl cyclohexane propionate | 3.935 |
| 2 | Ambrettolide | 6.261 |
| 3 | Amyl benzoate | 3.417 |
| 4 | Amyl cinnamate | 3.771 |
| 5 | Amyl cinnamic aldehyde | 4.324 |
| 6 | Amyl cinnamic aldehyde dimethyl acetal | 4.033 |
| 7 | Iso-amyl salicylate | 4.601 |
| 8 | AURANTIOL (Hydroxycitronellal- | 4.216 |
| | methylanthranilate) | |
| 9 | Benzyl salicylate | 4.383 |
| 10 | Para-tert-Butyl cyclohexyl acetate | 4.019 |
| 11 | Iso butyl quinoline | 4.193 |
| 12 | beta-Caryophyllene | 6.333 |
| 13 | Cadinene | 7.346 |
| 14 | Cedrol | 4.530 |
| 15 | Cedryl acetate | 5.436 |
| 16 | Cedryl formate | 5.070 |
| 17 | Cinnamyl cinnamate | 5.480 |
| 18 | Cyclohexyl salicylate | 5.265 |
| 19 | Cyclamen aldehyde | 3.680 |
| 20 | Diphenyl methane | 4.059 |
| 21 | Diphenyl oxide | 4.240 |
| 22 | Dodecalactone | 4.359 |
| 23 | ISO E SUPER (1-(1,2,3,4,5,6,7,8- | 3.455 |
| | Octahydro-2,3,8,8-tetramethyl-2- | |
| | naphthalenyl)-ethanone) | |
| 24 | Ethylene brassylate | 4.554 |
| 25 | Ethyl undecylenate | 4.888 |
| 26 | EXALTOLIDE (15-Hydroxyentadecanloic | 5.346 |
| | acid, lactone) | |
| 27 | GALAXOLIDE (1,3,4,6,7,8-Hexahydro- | 5.482 |
| | 4,6,6,7,8,8-hexamethylcyclopenta- | |
| | gamma-2-benzopyran) | |
| 28 | Geranyl anthranilate | 4.216 |
| 29 | Geranyl phenyl acetate | 5.233 |
| 30 | Hexadecanolide | 6.805 |
| 31 | Hexenyl salicylate | 4.716 |
| 32 | Hexyl cinnamic aldehyde | 5.473 |
| 33 | Hexyl salicylate | 5.260 |
| 34 | Alpha-Irone | 3.820 |
| 35 | LILIAL (para-tertiary-butyl-alpha- | 3.858 |
| | methyl hydrocinnamic aldehyde) | |
| 36 | Linalyl benzoate | 5.233 |
| 37 | Methyl dihydrojasmone | 4.843 |
| 38 | Gamma-n-Methyl ionone | 4.309 |
| 39 | Musk indanone | 5.458 |
| 40 | Musk tibetine | 3.831 |
| 41 | Oxahexadecanolide-10 | 4.336 |
| 42 | Oxahexadecanolide-11 | 4.336 |
| 43 | Patchouli alcohol | 4.530 |
| 44 | PHANTOLIDE (5-Acetyl-1,1,2,3,3,6- | 5.977 |
| | hexamethyl indan) | |
| 45 | Phenyl ethyl benzoate | 4.058 |
| 46 | Phenylethylphenylacetate | 3.767 |
| 47 | Phenyl heptanol | 3.478 |
| 48 | Alpha-Santalol | 3.800 |
| 49 | THIBETOLIDE (15-Hydroxypentadecanoic | 6.246 |
| | acid, lactone) | |
| 50 | Delta-Undecalactone | 3.830 |
| 51 | Gamma-Undecalactone | 4.140 |
| 52 | Vetiveryl acetate | 4.882 |
| 53 | Ylangene | 6.268 |
| 54 | Methyl Beta Napthyl Ketone | 1.99 |
| 55 | Terpeneol Couer | 2.67 |
| 56 | Geraniol | 2.7 |
| 57 | Dihydromyrcenol | 2.99 |
| 58 | Citronellol 950 | 3.3 |
| 59 | Tetrahydromyrcenol | 3.54 |

The higher ClogP materials are preferred, meaning that those materials with a ClogP value of 4.5 are preferred over those fragrance materials with a ClogP of 4.0; and those materials with a ClogP value of 4.0 are preferred over the fragrance materials with a ClogP of 3.3.

The fragrance formulation of the present invention preferably has at least about 40 weight percent of materials with ClogP greater than 3.3, preferably greater than about 80 and more preferably greater than about 90 weight percent of materials with ClogP greater than 4.0.

In an additional embodiment, the fragrance formulation may contain fragrance materials with a ClogP greater than about 1.5.

Those with skill in the art appreciate that fragrance formulations are frequently complex mixtures of many fragrance ingredients. A perfumer commonly has several thousand fragrance chemicals to work from. Those with skill in the art appreciate that the present invention may contain a single ingredient, but it is much more likely that the present invention will include at least eight or more fragrance chemicals, more likely to contain twelve or more and often twenty or more fragrance chemicals. The present invention also contemplates the use of complex fragrance formulations containing fifty or more fragrance chemicals, seventy-five or more, or even a hundred or more fragrance chemicals in a fragrance formulation.

Preferred fragrance materials will have both high ClogP and high vapor pressure. Among those having these properties are: Para cymene, Caphene, Mandarinal Firm, Vivaldie, Terpinene, Verdox, Fenchyl acetate, Cyclohexyl isovalerate, Manzanate, Myrcene, Herbavert, Isobutyl isobutyrate, Tetrahydrocitral, Ocimene and Caryophyllene.

Preferably, the fragrance or odorant or the mixture of fragrances or odorants makes up at least 50% by mass, preferably 60 to 90% by mass, or more preferably 70 to 80% by mass of the oil phase used in preparing the hydrogel capsule of this invention.

To provide the highest fragrance impact from the fragrance encapsulated capsules deposited on the various substrates referenced herein, it is preferred that materials with a high odor-activity be used. Materials with high odor-activity can be detected by sensory receptors at low concentrations in air, thus providing high fragrance perception from low levels of deposited capsules. This property must be balanced with the volatility as described herein. Some of the principles mentioned above are disclosed in U.S. Pat. No. 5,112,688.

Preferably, the fragrance or odorant or the mixture of fragrances or odorants makes up at least 50% by mass, preferably 60 to 90% by mass, or more preferably 70 to 80% by mass of the oil phase used in preparing the hydrogel capsule of this invention. In certain aspects, the active material is a fragrance oil and the total loading of the fragrance oil in the microcapsules is in the range of from 10% to 40% by weight of the microcapsule.

The polymerizable material used in the preparation of the microcapsules of this invention preferably includes at least one bio-degradable polymer. Bio-degradable polymers that can be added during the preparation of the microcapsules include, but are not limited to, sodium alginate, chitosan, tannic acid, or a combination thereof. In some aspects, the bio-degradable polymer used in the preparation of the instant microcapsules is a combination of chitosan and tannic acid.

“Chitosan” is a linear polysaccharide composed of randomly distributed β-(1-4)-linked D-glucosamine (deacetylated unit) and N-acetyl-D-glucosamine (acetylated unit). Chitosan can be generated from chitin obtained from crustaceans, insects or is more preferably obtained from a mushroom or fungal source or derived from fungal chitin by chemical deacetylation. Exemplary fungal sources that may be used in the preparation of chitosan include, but are not limited to, Pleurotus ostreatus and Aspergillus niger.

Advantageously, the incorporation of one or more bio-degradable polymers imparts the microcapsules with the capacity to undergo bio-degradation. In some aspects, the microcapsules shell is inherently bio-degradable, primarily bio-degradable or ultimately bio-degradable. In other aspects, the microcapsules shell has a bio-degradability of at least 20%, 30%, 40%, 50%, 60%, 65%, 70%, 75%, 80%, 85%, 90%, 95% or 98%, within 60 days according to OECD301F or OECD310.

The microcapsules of this invention are produced by (a) providing an aqueous phase, which contains sodium alginate, chitosan, tannic acid, or a combination thereof (b) providing an oil phase, which contains an active material, e.g., a fragrance or odorant; (c) emulsifying the aqueous phase of (a) with the oil phase of (b) to produce an emulsion; (d) polymerizing the emulsion to produce a microcapsule with a fragrance or odorant encapsulated therein; and (e) curing the hydrogel capsule.

The aqueous phase of the method includes water and one or more of sodium alginate, chitosan, and tannic acid. The aqueous phase may optionally include an emulsifier. An emulsifier is an agent used to bind together normally noncombinative substances, such as oil and water. It can be anionic, cationic or nonionic in nature. Examples of suitable emulsifiers include, but are not limited to, polyvinyl alcohol, e.g., a partially or completely hydrolyzed polyvinyl acetate (Biehn & Ernsberger (1948) Ind. Eng. Chem. 40:1449-1453), d-α-tocopheryl polyethylene glycol 1000 succinate (Mu & Feng (2003) Pharma. Res. 20:1864-1872), PLURACARE® or poloxamer, or polyvinyl pyrrolidone.

The oil phase of the method of this invention includes at least one active material as described herein and may optionally include an oil. Exemplary oils of use herein include ISOPAR® M (an isoparaffinic fluid) and the other ISOPAR® variants available from ExxonMobil Corp.; caprylic and capric triglycerides (e.g., NEOBEE® M-5, NEOBEE® M-20, triglycerides of coconut oil; and NEOBEE® 895, caprylic triglyceride, available from Stepan Chemicals), light mineral oils, light mineral waxes, vegetable oils, light vegetable waxes, diethylphthalate, butylbenzoate, benzylbenzoate, ester solvents, triacetin, and glycol-based water-insoluble solvents.

Once the oil phase and aqueous phase are combined, the mixture is emulsified according to known techniques, e.g., homogenization, shaking, or exposure to ultrasound. Subsequently, the wall material is polymerized to produce a microcapsule with a fragrance or odorant encapsulated therein. Polymerization can be carried out using one or more cross-linkers such as aliphatic polyisocyanates (e.g., a polyisocyanate based on hexamethylene diisocyanate) or an amino acid such as lysine. In some aspects, tannic acid is used as a cross-linker. In this respect, the tannic acid may be added to the mixture prior to and/or after emulsification to facilitate polymerization. Polymerization can be carried out at room temperature (e.g., 20-25° C.) for one to several hours.

The resulting microcapsules are subsequently cured, e.g., at an elevated temperature of at least 40° C. In certain aspects, the microcapsules are cured at temperature in the range of 55° C. and 65° C.

Microcapsules of this invention have a mean diameter in the range of 1 μm to 100 μm, more preferably in the range of 1 μm to 20 μm, most preferably in the range of 5 μm to 10 μm; and are stable during storage.

The microcapsules of this invention are also suitable for laundry care, personal care, therapeutic, cosmetic or cosmeceutic products. In particular, the microcapsules of this invention are of particular use in wash-off products, which are understood to be those products that are applied for a given period of time and then are removed. Such products include laundry care products such as fabric conditioners, liquid detergent, powder detergent, and fabric refresher; as well as personal care products such as hair shampoos, hair conditioners, hair rinses, antiperspirant/deodorant, hand sanitizer, bar soaps, and body washes and the like. These products are well known in the art. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, 4,767,547, and 4,424,134. Liquid dish detergents are described in U.S. Pat. Nos. 6,069,122 and 5,990,065; automatic dish detergent products are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562. Liquid laundry detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681.

Personal care products, including cosmetic, cosmeceutic or pharmaceutical preparations can be formulated as “water-in-oil” (W/O) type emulsions, “oil-in-water” (O/W) type emulsions or as multiple emulsions, for example of the water-in-oil-in-water (W/O/W) type, as a PIT emulsion, a Pickering emulsion, a micro-emulsion or nano-emulsion. Emulsions that are particularly preferred are of the “oil-in-water” (O/W) type or water-in-oil-in-water (W/O/W) type.

In certain embodiments, the final composition or product may be in the form of an oil, a gel, a roll-on, a solid stick, a lotion, a cream, a milk, an aerosol, a spray, a powder, a foam, a shampoo, a hair conditioner, a lacquer or a make-up.

The invention is described in greater detail by the following non-limiting examples.

### Example 1: Chitosan/Tannic Acid Microcapsules (Microcapsule 1)

An oil phase was first prepared by mixing 102 g of a model fragrance and 11.34 g of caprylic/capric triglyceride (a core solvent, commercially available under the tradename NEOBEE® oil M-5; Stepan, Chicago, Ill.). In a separate beaker, 457.11 g of an aqueous solution was obtained by dissolving fungal chitosan (9.0 g), gum acacia (a capsule formation aid, 6.0 g), and a polyoxyethylated castor oil (1.8 g, commercially available under the tradename TOXIMUL® 8240; Stepan, Chicago, Ill.) in water. The oil phase was then emulsified into the aqueous phase to form an oil-in-water emulsion under a shearing rate of 8600 rpm for three minutes.

The oil-in-water emulsion was prepared under constant mixing at room temperature. An aliphatic polyisocyanate (1.2 g) (a polyisocyanate based on hexamethylene diisocyanate (HDI) commercially available under the tradename DESMODUR® N100A; Bayer, Leverkusen, Germany) in ethyl acetate (4.5 g) (Sigma-Aldrich, St. Louis, Mo.) solution was added to the emulsion. The mixture was cured at room temperature for 30 minutes. Then, the mixture was cured at 60° C. for 1 hour. Subsequently, 10.8 g of 30% tannic acid aqueous solution (Tanal-02; Ajinomoto, Japan) was added under constant mixing. The slurry was cured for 2 hours at 60° C. L-lysine (4.131 g of a 15% solution; Sigma-Aldrich, St. Louis, Mo.) was added and the slurry was cured for an additional 1 hour to obtain Microcapsule 1 dispersed in the aqueous phase.

### Example 2: Chitosan/Carrageenan Microcapsules (Microcapsule 2)

An oil phase was first prepared by mixing 102 g of a model fragrance and 11.34 g of caprylic/capric triglyceride (a core solvent, commercially available under the tradename NEOBEE® oil M-5; Stepan, Chicago, Ill.). In a separate beaker, 457.11 g of an aqueous solution was obtained by dissolving fungal chitosan (9.0 g), gum acacia (a capsule formation aid, 6.0 g), Carrageenan A-CAT (IFF Nutrition and Biosciences, Wilmington Del.) (6.0 g), and a polyoxyethylated castor oil (1.8 g, commercially available under the trademark TOXIMUL® 8240; Stepan, Chicago, Ill.) in water. The oil phase was then emulsified into the aqueous phase to form an oil-in-water emulsion under a shearing rate of 8600 rpm for three minutes.

The oil-in-water emulsion was prepared under constant mixing at room temperature. An aliphatic polyisocyanate (1.2 g) (a polyisocyanate based on HDI commercially available under the tradename DESMODUR® N100A; Bayer, Leverkusen, Germany) in ethyl acetate (4.5 g) (Sigma-Aldrich, St. Louis, Mo.) solution was added to the emulsion. The mixture was cured at room temperature for 30 minutes. Subsequently, 6.0 g of a 30% tannic acid aqueous solution (Tanal-02; Ajinomoto, Japan) was added under constant mixing for 30 minutes. Lysine (9.3 g of a 15% solution; Sigma-Aldrich, St. Louis, Mo.) was added and the mixture was incubated for 30 minutes. The mixture was then heated to 60° C. Two portions of 6.0 g of 30% tannic acid aqueous solution (same as above) and two portions of 9.3 g of 15% L-Lysine (same as above) were added into the mixture, interactively; 30 minutes for each material/portion. In total, 18.0 grams of tannic acid and 27.9 grams of Lysine were added over a 2-hour period. The resulting slurry containing Microcapsule 2 was dispersed in the aqueous phase. The encapsulation efficiency was 99.9%.

### Example 3: Chitosan/Carrageenan Microcapsules (Microcapsule 3)

Microcapsule 3 was prepared following the procedure described in Example 2 except that after 2 hours curing time at 60° C., one extra hour curing at 80° C. was added.

### Example 4: Chitosan/Carrageenan Microcapsules (Microcapsules 4-6)

Microcapsules 4-6 were prepared by the same process described in Example 2 using the formulations provided in Table 2.

TABLE 2

| | Microcapsule | | |
|---|---|---|---|
| Ingredients (%) | 4 | 5 | 6 |
| Fungal Chitosan | 1.5 | 1.5 | 1.5 |
| Carrageenan | 1.0 | 1.0 | 1.0 |
| Gum Acacia | 1.0 | 1.0 | 1.0 |
| TOXIMUL ® 8240 | 0.3 | 0.3 | 0.3 |
| DESMODUR ® N100A | 0.2 | 0.1 | 0.05 |
| Tannic Acid | 0.9 | 0.9 | 0.9 |
| Lysine | 0.7 | 0.7 | 0.7 |
| Fragrance | 17.0 | 17.0 | 17.0 |
| NEOBEE ® | 1.9 | 1.9 | 1.9 |

### Example 5: Deodorant and Antiperspirant

An exemplary wax-based deodorant is prepared by mixing paraffin wax (10-20%), hydrocarbon was (5-10%), white petrolatum (10-15%), acetylated lanolin alcohol (2-4%), diisopropyl adipate (4-8%), Mineral Oil (40-60%) and preservative (as needed); heating the mixture to 75° C. until melted, and, with stirring at 75° C., adding 4.0 parts by weight of an encapsulated fragrance of this invention.

An exemplary glycol/soap type deodorant is prepared by combining propylene glycol (60-70%), sodium stearate (5-10%), distilled water (20-30%) and 2,4,4-trichloro-2′-hydroxy diphenyl ether (0.01-0.5%), and heating the mixture, with stirring, to 75° C. until the sodium stearate has dissolved. The resulting mixture is cooled to 40° C. and encapsulated fragrance is added to the formulation.

An exemplary antiperspirant deodorant (soft solid) is prepared by combining cyclopentasiloxane (60%), dimethicone (10%), zirconium aluminum trichlorohydrex glycine (25%), encapsulated fragrance (2.5%) and fumed silica (2.5%).

### Example 6: Body Wash

An exemplary body wash is composed of PLANTAPON® 611 L (SLES, Cap Betaine, Lauryl Glycoside; 22%), ammonium lauryl sulfate (2.5%), LAMESOFT® OP65 (Coco Glucoside, Glyceryl Oleate; 3%), polyquaternium 10-10 (0.5%), acrylates copolymer (0.5%), neat fragrance (0.3%), encapsulated fragrance (1%), DMDM hydantoin (0.3%), glycerin (3%) and water (q.s. 100%).

### Example 7: Hair Products

An exemplary 2-in-1 hair shampoo is composed of sodium laureth sulfate (10%), cocamidopropyl betaine (7%), glyceryl stearate (2%), cetearyl alcohol (3%), panthenol (0.2%), acrylates copolymer (1.2%), dimethicone (1.5%), polyquaternium 10 (0.2%), encapsulated fragrance (1%), preservative (as needed), water (q.s. 100%), and NaOH to pH 6.0.

An exemplary hair gel is composed of PVP (3%), acrylates/C10-30 alkyl methacrylate copolymer (3%), denatured alcohol (10%), encapsulated fragrance (1%), Microcare PHG (0.5%), and water (q.s. 100%).

### Example 8: Hand Sanitizer

An exemplary hand sanitizer is composed of acrylates C10-30 alkyl acrylate copolymer (0.2-0.5%), ethanol (60%), isopropanol (10%), glycerin (4%), encapsulated fragrance (1-5%), and water (q.s. 100%).

What is claimed is:

1. Bio-degradable core-shell microcapsules comprising
(a) a shell comprising sodium alginate, chitosan, tannic acid or a combination thereof; and
(b) a core comprising an active material.

2. The bio-degradable core-shell microcapsules of claim 1, wherein the active material comprises a fragrance or odorant.

3. The bio-degradable core-shell microcapsules of claim 1, wherein the microcapsules shell has a bio-degradability of at least 20%, 30%, 40%, 50%, 60%, 65%, 70%, 75%, 80%, 85%, 90%, 95% or 98%, within 60 days according to OECD301F or OECD310.

4. The bio-degradable core-shell microcapsules of claim 1, wherein the microcapsules shell is inherently bio-degradable, primarily bio-degradable or ultimately bio-degradable.

5. The bio-degradable core-shell microcapsules of claim 1, wherein the active material is a fragrance oil and has a total loading that ranges from 10% to 40%.

6. The bio-degradable core-shell microcapsules of claim 1, wherein the chitosan is fungal-derived chitosan or a derivative thereof.

7. A method for making bio-degradable core-shell microcapsules comprising:
(a) preparing an aqueous phase comprising sodium alginate, chitosan, tannic acid or a combination thereof;
(b) preparing an oil phase containing an active material;
(c) emulsifying the aqueous phase and oil phase to form an emulsion;
(d) polymerizing the emulsion to produce bio-degradable core-shell microcapsules with the active material encapsulated therein; and
(e) curing the bio-degradable core-shell microcapsules at a temperature in the range of 55° C. to 95° C.

8. The method of claim 7, wherein the active material comprises a fragrance or odorant.

9. Bio-degradable core-shell microcapsules prepared by the method of claim 7.

10. A consumer product comprising the bio-degradable core-shell microcapsules of claim 1.

11. The consumer product of claim 10, wherein said consumer product is a laundry care, personal care, therapeutic, cosmetic or cosmeceutical product.

12. The consumer product of claim 11, wherein the personal care product is a hair shampoo, hair conditioner, hair rinse, antiperspirant deodorant, hand sanitizer, bar soap or body wash.

13. The consumer product of claim 11, wherein the personal care product is formatted as a stick, roll-on or aerosol spray.

14. The consumer product of claim 11, wherein the laundry care product is a fabric conditioner, liquid detergent, powder detergent or fabric refresher.
